(19) United States
(12) Patent Application Publication
(10) Pub. No.: US 2007/0073216 A1
(43) Pub. Date: Mar. 29, 2007

(54) Method and apparatus for delivering a colonic lavage

(75) Inventors: Anthony Carlisle McAuliffe, Lilburn, GA (US); Michael Calvin Hardy, Lawrenceville, GA (US)

Correspondence Address: Anthony McAuliffe, 3468 Runelle Place, Lilburn, GA, 30047, US

(73) Assignee: The Pulsed Enema Company

(21) Appl. No.: 11/175,234

(22) Filed: Jul. 7, 2005

(51) Int. Cl. A61M 1/00; A61M 31/00

(52) U.S. Cl. 604/30; 604/514

(57) ABSTRACT

A portable, lightweight bowel evacuation apparatus and method of delivering a colonic lavage is provided. The apparatus includes a liquid bag containing lavage liquid connected to a drain delivery line that leads to a rectal insertion tube or speculum. The apparatus also includes a drain line leading from the speculum to a waste collection. A pressurizing member, such as a manual pump or squeeze bulb is provided to generate pulsed waves in the lavage liquid in the patient's colon so as to break up and remove fecal material.

### CROSS-REFERENCE TO RELATED APPLICATIONS

This application claims the benefits of the filing date of the U.S. Provisional Patent Application Ser. No. 60/585,533, filed on Jul. 6, 2004, and which is incorporated by references as if fully set forth in its entirety herein.

### TECHNICAL FIELD

This invention relates generally to devices and methods of delivering a colonic lavage, to manual pumps usable in such devices and methods, and to a pressure limited manual control valve usable is such devices and methods.

### BACKGROUND

In the home and long-term care settings, if an individual is incapacitated and can not mount a bedpan, bedside toilet chair, or a bathroom toilet, the individual usually does not have an opportunity to evacuate on a regular basis. This lack of opportunity can lead to serious fecal impactions that require medical attention. Traditionally, individuals in home care and long-term care facilities have been required to seek treatment for the fecal impactions in hospitals. Usually these hospital visits entail at least a three day visit admission, barrages of oral administered chemicals, series of ordinary enemas, and often digital removal of the stool using a rubber glove. If these procedures fail, then surgery has been the remaining alternative.

Traditional enemas also are employed frequently in home and long-term care facilities to facilitate proper bowel evacuation. Unfortunately, ordinary enemas often result in inadequate bowel evacuations and their associated problems. Generally, conventional enemas provide for an infusion of water into the individual's colon, but do not provide a means of collecting the wastewater and waste material. During the infusion step, the patient will usually experience cramping in their colon, necessitating stopping the procedure to allow the patient to void. After receiving the infusion of water from an ordinary enema, the patient must quickly mount a bedpan, bedside toilet chair, or bathroom toilet in order to dispose of the waste material. Not surprisingly, this step often results in bowels accidents that leave waste material on the bed or floor. As a result, many patients will elect to sit on the toilet while the ordinary enema is administered. However, this delivery position can be harmful to the patient, since the colon tends to be vertically aligned and the waste pressure pressing sideways on the colon walls can cause the colon to enlarge.

Evan after contending with these disadvantages, the patient's colon often is not completely cleaned because ordinary enemas usually cannot break up and remove fecal impactions or completely clean the colon in a reasonable amount of time, due in part to the fact that most conventional enemas include water reservoirs that can hold only about one liter of fluid. Accordingly, patients with limited mobility often can have bowel accidents after an enema, leaving them to lie or sit in their own fecal material. Medical studies have revealed that patients lying in their own stool for extended periods of time are susceptible to the occurrence of urinary tract infections, dermatological infections and decubitus ulcers, which often require hospital visits or surgery to treat.

While there are some alternatives to the conventional enema currently available, these conventional devices generally are burdened with various problems and shortcomings of their own. For example, many of the devices are large, bulky, and heavy and suited for use only by medical personnel in the confines of a doctor's office, hospital, or other appropriate facility. Most are not suitable for personal use by a patient in his or her home, and certainly cannot be carried easily on trips away from home. In addition, many conventional devices include pumping and valve mechanisms that tend to deliver lavage liquid to the colon in a harsh manner that can be uncomfortable for the patient and ineffective in removing certain types of fecal impactions. Most of these devices include valves and specula that are formed of hard, uncomfortable components and include injection molded plastics parts, which are relatively expensive, especially for single use disposable items.

Consequently, there is a need for a lightweight, self contained, reliable, integrated apparatus for delivering a colonic lavage. The apparatus should be easily transported, be made with highly reliable yet economical valves, speculums, and receptacles that can be used once and thrown away.

### SUMMARY

The present invention generally encompasses a method and apparatus for delivering a colonic lavage with pulsating action produced by a manual pump. In one embodiment, the apparatus comprises a water reservoir connected to a delivery line and cutoff valve or clamp for opening and closing the delivery line, a rectal insertion tube in communication with the delivery line for directing water from the water reservoir into the colon, a drain line in communication with the tube, including a pressure limited drain valve or cutoff for selectively opening and closing the drain line, and a manual pump or squeeze bulb in flow communication with the interior of the rectal insertion tube for pressurizing or pulsing water delivered to the colon so as to generate waves to displace impactions and other fecal material. The manual pump can be in-line with the delivery line, the drain line, or can be arranged in a separate line in flow communication with the rectal insertion tube and water inlet.

In one embodiment, the bowel evacuation apparatus comprises a fresh liquid bag that serves as the liquid reservoir. The bag includes a handle that can be used for carrying the bag and for suspending it above the patient to provide the liquid by gravity feed. The bag is in flow communication with a delivery line that has a clamp attached thereto for opening and closing the line. A manual pump or squeeze bulb is disposed in-line with the delivery line between the fresh liquid bag and a rectal insertion tube. The delivery line is connected to an inlet of a three port connector. The three port connector is disposed in-line to the rectal insertion tube or speculum having an opening therein that is in flow communication with the user's colon. A drain line is connected to the three port connector outlet and leads to a waste reservoir, such as a bag. The drain line could also lead directly into a commode or toilet. A drain valve is connected to the drain line, so that when water is being delivered to the colon, the drain line is closed. The drain valve is a three port connector. The drain valve connector is lined with a silicone tube or similar material entering and exiting the two straight ports of the drain valve connector. The silicone is sealed over the outside edges of the drain valve connector ports forming a liner between the in-line ports of the drain valve connector. The third port is attached to a drain valve inflation line. The drain valve inflation line is connected to an inflation squeeze bulb or other inflation device. The drain valve inflation line also contains an air check valve. The air check valve limits the amount of pressure on the drain valve. The drain valve acts as a safety feature and limits the water pressure on the colon. The drain valve is closed by initiating the drain inflation device and creating air pressure upon the silicone in the drain valve connector. The air pressure on the silicone liner forces the liner to expand and close within the drain valve connector. The drain valve described above could be replaced by a manual ratchet clamp.

Those parts of the system which are exposed to waste materials, including the delivery line, manual pump, rectal insertions tube, drain line and collection bag may be replaced when desired with out replacing the other parts of the system. The entire system can be extremely lightweight, compact and conveniently stored when desired.

The present invention also encompasses a method of administering a colonic lavage in which liquid introduced into the colon is pulsed to provide desired agitation that tends to break up fecal matter and impactions. The method generally includes delivering a lavage liquid into a patient's colon, pressurizing the liquid by manual means, and then emptying the liquid from the patient's colon. In one embodiment, the method also comprises charging a liquid reservoir with lavage liquid, opening a fresh liquid clamp to allow liquid to enter the patient's colon, closing a drain valve to maintain the liquid in the patient's colon and squeezing a manual pump to pulse the lavage liquid. The method also can include inserting a speculum into the patient's rectum and inflating a cuff to retain the speculum in the patient's rectum. Furthermore, the method can include collecting waste material in a waste collector. The steps of the method can be carried out once or repeatedly to provide a cleansing colonic lavage.

These and other aspects of the present invention are described below and shown in the drawings which are briefly described as follows.

### BRIEF DESCRIPTION OF THE DRAWINGS

FIG. 1 is a schematic illustration of a colonic lavage assembly encompassing principles of the present invention.

FIG. 2 is a perspective view of a speculum that can be used in the colonic lavage assembly of FIG. 1.

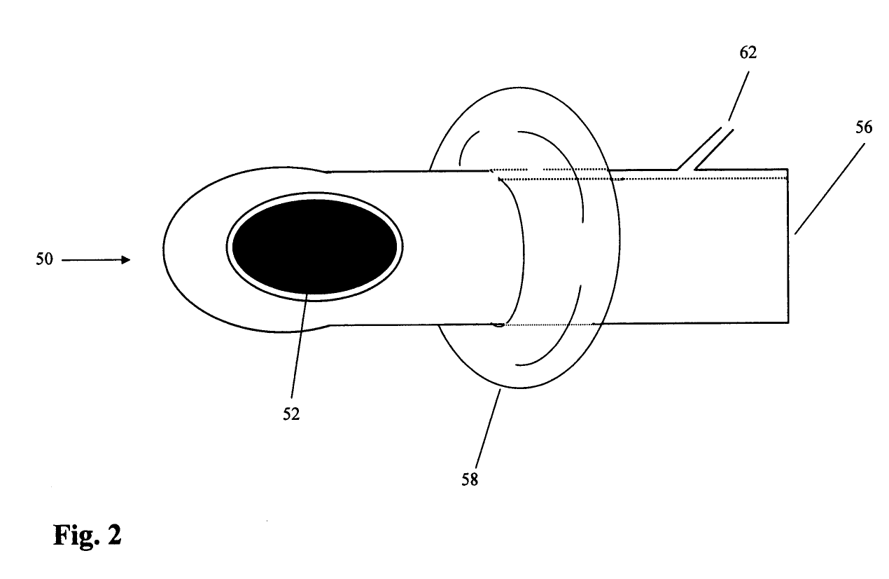

FIG. 3 is a perspective view of a drain valve that can be used in the colonic lavage assembly of FIG. 1.

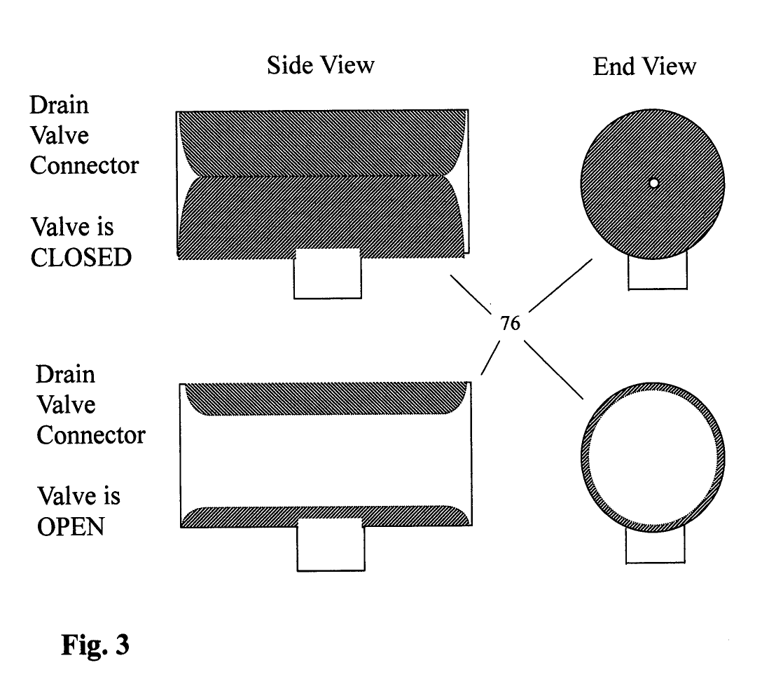

### DETAILED DESCRIPTION

Referring now to the drawings, a colonic lavage liquid delivery and waste removal apparatus 100 is provided. The apparatus 100 is designed to provide a colonic lavage with the capability of providing manually controlled pulsation to the lavage liquid through use of a manual pump or squeeze bulb 44 that can be inline with the fresh liquid delivery line 30. Since the apparatus 100 includes both a liquid reservoir and a waste receptacle, the patient need not move in order to remove fecal material from the colon. Rather, a colonic lavage or pulsing enema can be delivered to a patient while the patient is on their back or left side, thereby avoiding placing excessive pressure on the walls of the patient's colon.

As shown in the figures, the apparatus 100 comprises a speculum 50 that is configured and sized to be inserted into a patient's rectum for delivering a colonic lavage. The speculum 50, which can be molded from a unitary piece of rubberized material such as silicone, or produced from vinyl tubing, has a generally tubular body with a closed bulbous distal end. A pair of oblong openings 52 are formed in the distal end of the speculum 50 for delivering lavage liquid to a colon and receiving waste liquid for extraction from the colon. A selectively inflatable cuff 58 is provided on the tubular body portion of the speculum 50 and can be selectively inflated and deflated by use of a inflation bulb 60. The inflation bulb 60 is in flow communication with an air inlet line 64, an air inlet line clamp 66, and an air inlet port 62 on the speculum 50. The air inlet port 62 ends under the inflatable cuff 58. The inflatable cuff 58 includes one layer of silicone tubing that has been folded over itself and glued at the joining ends for a complete air enclosure to create a balloon. A small hole is created in the bottom layer of the balloon to allow air to enter the balloon from the air inlet port 62 on the side of the speculum 50. An adhesive, disposed between the inflatable cuff 58 and the speculum 50, prevents air leakage. The speculum 50 has an inlet port 56. The present invention can be utilized without the inflatable cuff 58.

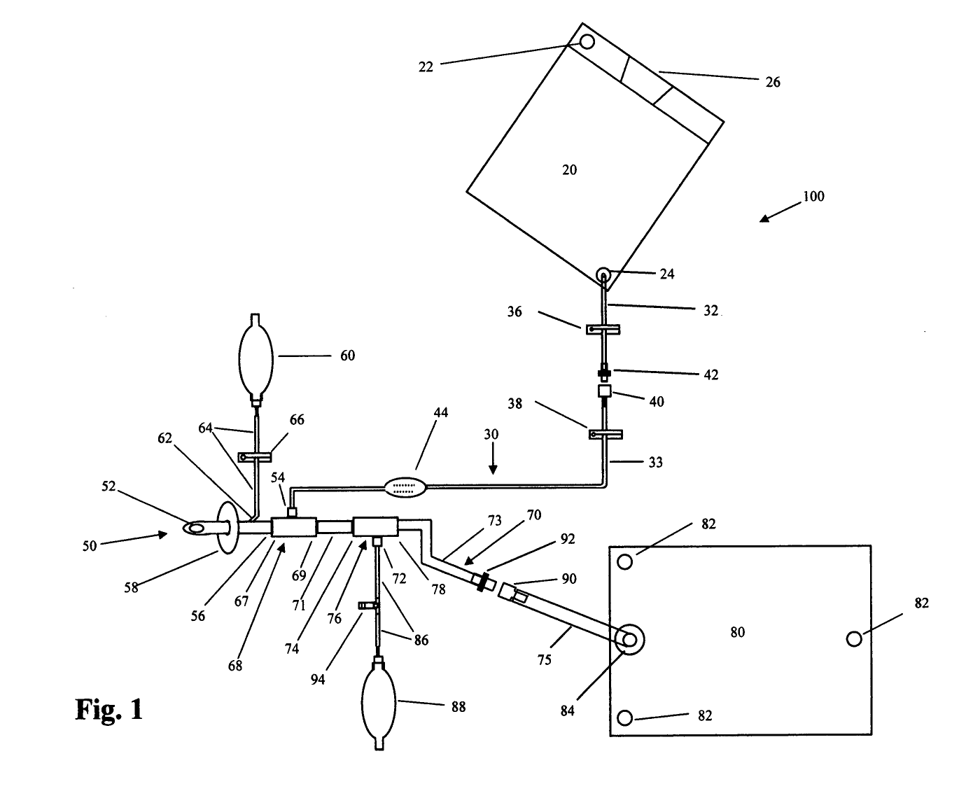

A delivery line 30 is secured to inlet port 54 of the delivery line connector 68. The delivery line 30 is composed of a first section 32 and a second section 33. A manual pump or squeeze bulb 44 is disposed in-line between the port 54 of connector 68 and the female connector 40. Male connector 42 and female connector 40 interconnect the first and second sections 32 and 33. The first section 32 of the delivery line 30 is connected to a connector 24 on a fresh liquid reservoir or bag 20. A delivery line shut-off valve or clamp 38 is operably connected to the delivery line 30 so as to allow liquid to flow from the fresh liquid bag 20 to the speculum 50. As shown in FIG. 1, clamp 38 can be disposed between the squeeze bulb 44 and the fresh liquid bag 20 to allow liquid delivered to the patient's colon through the speculum to flow back to the squeeze bulb 44, but not to the fresh liquid bag 20. The fresh liquid bag 20 can be formed of a polymeric material such as polyethylene, polypropylene, or rubber. Likewise, the delivery line 30 can be formed of a polymeric material that can be collapsed and sealed with clamp 38. A second delivery line shut-off valve or clamp 36 can be disposed between the male connector 42 and the fresh liquid bag 20 so as to allow the disconnection of the male connector 42 and female connector 40, when clamp 36 is closed, and not allow liquid to flow from fresh liquid bag 20.

While water is usually the liquid chosen to be delivered to a patient's colon, other liquids, medications, and materials, may be delivered using the apparatus of the present invention. The fresh liquid bag 20 has a handle 22 or other suspension member that can be used to hang the bag from a stand, not shown, at an elevated height above the patient for gravity flow of water from the bag 20. Typically, the liquid bag 20 is placed approximately 2′ over a bed or other surface upon which the patient lies. Clamp 38 is positioned on the delivery line 30 leading from an outlet port 24 at the lower end of the bag, clamp 38 controlling the flow or delivery of water from the liquid bag 20. In an alternative embodiment, a check valve, not shown, may be disposed in line with the delivery line 30 or the squeeze bulb 44. The check valve can allow the squeeze bulb 44 to be used to pump liquid from the fresh liquid bag 20 into the speculum 50, thereby providing an alternative to gravity feed. Once the desired amount of liquid is delivered to the patient's colon, the check valve can be closed and the squeeze bulb 44 operated as set forth below.

A drain line 70 is secured to port 69 of the delivery line connector 68. The drain line 70 is composed of a first section 71, a second section 73, and a third section 75. Male connector 92 and female connector 90 interconnect the second and third sections 73 and 75. The first section 71 is disposed between port 69 of the delivery line connector 68 and port 74 of the drain valve 76. The second section is disposed between port 78 of the drain valve 76 and the male connector 92. The third section 75 of the drain line 70 disposed between female connector 90 and connector 84 on waste bag 80.

The speculum 50 is secured at its open back end or outlet 56 to port 67 of the delivery line connector 68. The port 69 of the delivery line connector 68 is secured to the drain line 70. The drain line 70 communicates between the speculum 50 and the waste liquid receptacle or waste bag 80, which may comprise a polymeric bag. A drain valve 76 is operably connected to the drain line 70 so as to regulate the flow of liquid between the speculum 50 and the waste bag 80. The drain valve 76 is a three port connector. The drain valve 76 connector is lined with a silicone tube entering and exiting the two straight ports 74 and 78 of the drain valve 76 connector. The silicone tube is sealed over the outside edges of the drain valve 76 connector ports 74 and 78. The third drain valve port 72 is attached to a drain valve inflation line 86. The drain valve inflation line 86 is connected to an inflation squeeze bulb 88 or other inflation device. The drain valve inflation line 86 also contains an air check valve 94. The drain line 70 can be constructed of a collapsible polymeric material, such as polyethylene, polypropylene, or rubber. It will be understood that, with this configuration, when the drain valve 76 is in its open configuration, waste liquid is free to flow through the outlet 56 in the speculum 50, through the drain line 70, and to the waste liquid receptacle 80. Conversely, when the drain valve 76 is closed, this path is blocked and the waste liquid does not flow out of a patient's colon. Both the clamp 38 and drain valve 76 are manually operated for simplicity and to allow for the economical manufacture of the assembly 100.

In practice, the fresh liquid bag 20 is filled with body temperature water or other suitable liquid and then is either suspended in an elevated position above the patient or placed on the bed or chair adjacent to the patient. The female connector 40 connected to the first section 32 of the delivery line 30 can be used as a funnel to fill the liquid bag 20. In one embodiment, the fresh liquid bag 20 is designed to hold approximately 2.5 gallons of liquid although the present invention also encompasses an apparatus capable of holding alternative amounts. The fresh liquid bag 20 is filled while the clamp 36 is open for the purposes of allowing the water flow through the first section 32 of the delivery line 30 into the fresh liquid bag 20. After the fresh liquid bag 20 is filled, the clamp 36 is closed until the water is to be delivered to the speculum 50 from the fresh liquid bag 20. The fresh liquid bag 20 could also utilize a flap valve 26 to fill the fresh liquid bag 20. The fresh liquid bag 20 is then placed on the bed/surface or hung from a hook/IV stand positioning the fresh liquid bag 20 above the level of the patient for gravity flow of the lavage liquid. The height of the fresh liquid bag 20 is such that the pressure level of water when released from the bag is sufficient to flow through the delivery line 30, squeeze bulb 44 and speculum 50 into the colon of the patient. The female connector 40 is then connected to the male connector 42 attached to the squeeze bulb 44.

The tip of the speculum 50 is lubricated with a lubrication jelly and inserted into the rectum of the patient. The speculum 50 is inserted into the patient's rectum up to the collar or anal ring, and the cuff 58 is inflated to an extent sufficient to prevent the speculum 50 from accidentally slipping out of the rectum during the bowel evacuation procedure. The inflation bulb 60 is repeatedly compressed to inflate the speculum cuff 58 sufficiently to fill the rectal vault of the patient. Once inflated to the desired extent, the air line is closed off with an air inlet line clamp 66 to maintain the cuff 58 in the inflated condition. This provides a securing method of holding the speculum 50 in the rectal vault of the patient preventing discharge of the speculum 50 during the procedure. This process also prevents water leakage between the speculum 50 and the rectal walls. The waste bag 80 is placed on the floor near the bed/surface of the patient. Placing the waste bag 80 on the floor provides a gravity flow of the waste water from the patient to flow into the waste bag 80 without any suction being generated that could possibly harm the patient.

The drain valve 76 is closed so as to prevent liquid from draining from the speculum 50. Clamp 38 is then opened to allow liquid to flow through the delivery line 30 into the speculum 50 and the patient's colon. Fresh water begins to flow by gravity into the patient's colon. An alternative to suspending the liquid bag 20 is to place the bag on the bed or a surface and gently compress the bag with hand pressure so as to move the water from the bag into the patient. When the desired amount of fresh water is infused into the patient, clamp 38 is closed.

With both the clamp 38 and drain valve 76 closed, an assistant or the patient begins to compress the squeeze bulb 44 to pressurize the liquid and to cause a pulse of liquid to enter the colon of the patient and then to return to and refill the squeeze bulb with liquid. Repeating this procedure of squeezing the squeeze bulb 44 causes the liquid to undulate in waves into and out of the patient, thereby tending to break up the fecal material in the patient's colon. It has also been found through clinical trials that pulsing of the lavage liquid triggers autonomic peristaltic action in the patient's colon, which further loosens and breaks up impacted waste material.

Upon ending the pulsing process, the drain valve 76 is opened, allowing the waste liquid and stool to exit the speculum 50 and to flow into the drain line 70 and the waste bag 80. After the waste water exits the patient's colon and enters the waste bag 80, the entire process of introducing fresh liquid and operating the squeeze bulb 44 can be repeated. This process is continued until it is determined that sufficient stool has been removed from the patient. Viewing the waste collected in the waste bag 80 is one way of determining the amount of stool that has been removed. The waste bag 80 can be detached from the rest of the apparatus 100, such as by disconnecting the male connector 92 from the female connector 90, and carried to a toilet for emptying. The handle 82 of the waste bag 80 is provided to facilitate the transport of the waste bag 80 or for suspending it in a convenient location. The process generally provides for the repeated infusion of pulsing water into the patient's colon leading to re-hydration and break-up of the stool material into particles small enough to exit the colon through the speculum 50 and flow into the waste bag 80.

The construction and arrangement of the elements making up the assembly 100 are such that the speculum 50, delivery line 30, squeeze bulb 44, fresh liquid bag 20, drain line 70 and waste bag 80 are disposable and can be removed or disconnected from the other elements of the system and replaced without disturbing or affecting the other elements of the system.

The invention has been described herein in terms of particular embodiments and methodologies. It will be apparent to those of skill in the art, however, that various modifications might well be made to the illustrated embodiments within the scope of the invention. For example, while silicone, a silicone compound, or vinyl tubing has been illustrated as one material from which the speculum is molded, other appropriate materials can be substituted. Further, the size and relative dimensions of the various components of the speculum, bags and lines can be different from those illustrated in the drawings depending upon the particular purpose for which it is intended. For example, the manual pump or squeeze bulb can be differently shaped or sized providing a more pronounced pulsed action. Also, the manual pump can be positioned inline with the drain line of the assembly or in flow communication with the speculum through a line that is separated from both the delivery line and the drain line. Finally, the apparatus for delivering a colonic lavage and its various components as described herein may well be constructed with a wide variety of shapes and configurations that could differ from the illustrated embodiments. These and other additions, deletions, and modifications might well be made to the disclosed embodiments by those of skill in the art without departing from the spirit and scope of the invention as set forth in the claims.

What is claimed is:

1. An apparatus for delivering a colonic lavage comprising:
a lavage liquid reservoir;
a delivery line in fluid communication with said lavage liquid reservoir;
a delivery line connector in fluid communication with delivery line;
a speculum having a mouth and tip formed therein, wherein said delivery line connector is in fluid communication with said mouth;
a manual pump in fluid communication with delivery line;
a drain line in fluid communication with said delivery line connector; and,
a waste receptacle in fluid communication with said drain line.

2. The apparatus of claim 1, wherein said manual pump is disposed in-line with said delivery line.

3. The apparatus of claim 1, further comprising a first clamp for regulating fluid flow between said lavage liquid reservoir and said mouth.

4. The apparatus of claim 1, further comprising of a releasable connector in fluid communication with said delivery line.

5. The apparatus of claim 1, wherein said lavage liquid reservoir is releaseably connected to said delivery line.

6. The apparatus of claim 3, wherein first said clamp is disposed between said releasable connector and said liquid lavage reservoir.

7. The apparatus of claim 1, further comprising a second clamp for regulating fluid flow between said liquid reservoir and said mouth.

8. The apparatus of claim 7, wherein said second clamp is disposed between said releasable connector and said manual pump.

9. The apparatus of claim 1, further comprising of a delivery line connector in fluid communication with said delivery line, said waste line, and said mouth.

10. The apparatus of claim 1, further comprising of a waste receptacle in fluid communication with said drain line.

11. The apparatus of claim 1, further comprising a drain clamp disposed between said waste receptacle and said mouth.

12. The apparatus of claim 11, further comprising of an inner cuff formed inside said pressure sensitive drain valve.

13. The apparatus of claim 10, wherein said waste receptacle is releaseably connected to said drain line.

14. The apparatus of claim 13, wherein releaseable connector is disposed between said drain clamp and said waste receptacle.

15. The apparatus of claim 1, further comprising a handle attached to said lavage liquid reservoir.

16. The apparatus of claim 1, further comprising a handle attached to said waste receptacle.

17. The apparatus of claim 1, further comprising an inflatable cuff formed on said speculum.

18. The apparatus of claim 17, further comprising a cuff pump in fluid communication with said inflatable cuff.

19. The apparatus of claim 17, further comprising of a line clamp disposed between said cuff pump and said inflatable cuff.

20. An apparatus for delivering a colonic lavage comprising:
a lavage liquid reservoir;
a delivery line in fluid communication with said lavage liquid reservoir;
a delivery line connector in fluid communication with delivery line;
a speculum having a mouth and tip formed therein, wherein said delivery line connector is in fluid communication with said mouth;
a manual pump in fluid communication with delivery line;
a drain line in fluid communication with said delivery line connector;
a pressure sensitive drain valve in fluid communication with drain line; and,
a waste receptacle in fluid communication with said drain line.

21. The apparatus of claim 20, wherein said manual pump is disposed in-line with said delivery line.

22. The apparatus of claim 20, further comprising a first clamp for regulating fluid flow between said lavage liquid reservoir and said mouth.

23. The apparatus of claim 20, further comprising of a releasable connector in fluid communication with said delivery line.

24. The apparatus of claim 20, wherein said lavage liquid reservoir is releasebly connected to said delivery line.

25. The apparatus of claim 22, wherein first said clamp is disposed between said releasable connector and said liquid lavage reservoir.

26. The apparatus of claim 20, further comprising a second clamp for regulating fluid flow between said liquid reservoir and said mouth.

27. The apparatus of claim 26, wherein said second clamp is disposed between said releasable connector and said manual pump.

28. The apparatus of claim 20, further comprising of a delivery line connector in fluid communication with said delivery line, said waste line, and said mouth.

29. The apparatus of claim 20, further comprising of a waste receptacle in fluid communication with said drain line.

30. The apparatus of claim 20, further comprising a pressure sensitive drain valve disposed between said waste receptacle and said mouth.

31. The apparatus of claim 30, further comprising of an inner cuff formed inside said pressure sensitive drain valve.

32. The apparatus of claim 31, further comprising a pressure sensitive drain valve manual pump in fluid communication with said pressure sensitive drain valve.

33. The apparatus of claim 30, further comprising of a air check valve in fluid communication and disposed between said pressure sensitive drain valve and said pressure sensitive drain valve manual pump.

34. The apparatus of claim 29, wherein said waste receptacle is releaseably connected to said drain line.

35. The apparatus of claim 34, wherein releaseable connector is disposed between aid pressure sensitive drain valve and said waste receptacle.

36. The apparatus of claim 20, further comprising a handle attached to said lavage liquid reservoir.

37. The apparatus of claim 20, further comprising a handle attached to said waste receptacle.

38. The apparatus of claim 20, further comprising an inflatable cuff formed on said speculum.

39. The apparatus of claim 38, further comprising a cuff pump in fluid communication with said inflatable cuff.

40. The apparatus of claim 38, further comprising of a line clamp disposed between said cuff pump and said inflatable cuff.

41. A method for delivering a colonic lavage comprising:
inserting a speculum into a rectum of a patient;
delivering a lavage liquid through the speculum to a colon of a patient;
pulsating the lavage liquid with a manual pump; and,
draining the lavage liquid from the colon into a waste receptacle.

42. The method of claim 41, further comprising closing the first clamp and pressure sensitive drain valve to maintain lavage liquid in the colon of the patient.

43. The method of claim 41, further comprising pulsing the lavage liquid in the colon of the patient with the manual pump.

44. The method of claim 41, further comprising opening the pressure sensitive drain valve to drain lavage liquid from the colon of the patient into the waste receptacle.

45. The method of claim 41, further comprising inflating a cuff to secure the speculum in the rectum of the patient.
